(19) United States
(12) Patent Application Publication
(10) Pub. No.: US 2005/0042609 A1
(43) Pub. Date: Feb. 24, 2005

(54) Method and system for detecting inter-chromosomal imbalance by fluorescent in situ hybridization (fish) on interphase nuclei

(75) Inventors: Francoise Soussaline, Paris (FR); Bernard Malfoy, Paris (FR); Marie Noelle Guilly, Paris (FR); Khuong Truong, Paris (FR); Bernard Dutrillaux, Paris (FR)

Correspondence Address: SUGHRUE MION, PLLC, 2100 PENNSYLVANIA AVENUE, N.W., SUITE 800, WASHINGTON, DC, 20037, US

(73) Assignee: (1) Imstar Image et Modelisation: Strategie Analyse et Realisation (2) Institut Curie (et al), Paris (FR)

(21) Appl. No.: 10/482,809

(22) PCT Filed: Jul. 3, 2002
(86) PCT No.: PCT/FR02/02317

(30) Foreign Application Priority Data
Jul. 3, 2001 (FR) 01/08803

(51) Int. Cl. C12Q 1/68; C07H 21/04

(52) U.S. Cl. 435/6; 536/25.32

(57) ABSTRACT

The invention concerns a system for detecting chromosomal imbalance by in situ hybridization of fluorescent probes on interphase nuclei, comprising the following phases: hybridizing in situ fluorescent probes on two separate chromosomes; exposing each probe with a different fluorochrome; measuring the intensity signals corresponding respectively to each probe thus exposed, on an assembly of nuclei, said measurement being carried out within a control cell population and within a cell population subjected to detection; calculating a ratio between the signals corresponding to each of the probes, said ratio calculation being carried out on said control cell population to provide a reference ratio and said population subjected to detection; comparing the fluorescence ratio corresponding to the population subjected to detection with the reference ratio; and processing the result of said comparison to detect an inter-chromosomal imbalance.

The present invention relates to a method for detecting interchromosomal imbalance by fluorescent in situ hybridization (FISH) on interphase nuclei. It also relates to a system using this method.

The most recurrent constitutional chromosomal imbalances found in newborn babies concern chromosomes 21, 18 and 13 and the sex chromosomes. Trisomy 21 (Down's syndrome) is the most recurrent abnormality: approximately 1.7 children in 1000 are affected (Stoll et al, 1998).

The number of cases of trisomy 21 increases with the age of the mother, in particular after 35 years of age, and a prenatal examination is advised in cases of late maternity. There is a current tendency toward late maternity: the percentage of women having a child while over the age of 35 has gone from 8% to 19% in four years (Stoll et al, 1994). It can be estimated that this percentage will increase further, but as of now, since the number of births in France is 750 000 per year, the need for trisomy 21 tests is, potentially, of the order of 150 000 per year.

Since the 1970s, prenatal diagnosis after amniocentesis has generally been carried out using conventional cytogenetics, which makes it possible to establish karyotypes on metaphase chromosomes and thus to detect an abnormal number of chromosomes. The relatively long period of time between the taking of the sample and the result of the diagnosis (2 weeks) constitutes the main drawback of this technique. In addition, the obtaining of usable metaphases is not certain in 100% of cases (1% failures) and false negatives can appear due to the presence of maternal cells (Verma et al, 1998).

More recently, fluorescent in situ hybridization (FISH) has made it possible to work directly on interphase nuclei without having to resort to cell culture. This technique consists in labeling chromosomes (see, for example, U.S. Pat. No. 5,756,696) or chromosomal regions using fluorescent probes and then counting the signals, i.e. the chromosomes emitting fluorescent signals (Pierluigi et al, 1996; Steinborn et al, 1996; Wei et al, 1997; Eiben et al, 1999). It is thus possible to obtain results in quite a short period of time (a few days) and the analysis can be carried out on a small number of sampled cells (a few hundred), whereas the cell culture requires a greater number of cells (from a few tens of thousands and upwards).

Various types of probes have been used for these studies: probes covering the centromeric region or regions of the long arm of chromosome 21 (cosmids, YACs) (Soloviev et al, 1995, Van Opstad et al, 1995; Pierluigi et al, 1996; Steinborn et al, 1996). Partial labeling of chromosome 21 with these probes makes it possible to evaluate the number of copies. It is therefore necessary to carry out manual or automatic signal counting in order to detect the cells having three signals corresponding to chromosome 21.

Several studies show the technical feasibility of in situ hybridization of fetal cells. However, there are few studies which give results of direct comparison between the FISH technique and diagnosis by conventional cytogenetics (Pierluigi et al, 1996; Steinborn et al, 1996; Wei et al, 1997; Eiben, et al, 1999; Thein et al, 2000). The authors of those studies mention agreements of 97-100% between the two approaches, but also underline the difficulties in interpreting the results with interphase FISH. These difficulties are essentially linked to the need for a human operator who recognizes and counts the fluorescent signals. Now, the undoubted variability between different operators and especially the statistical significance of the number of nuclei with an abnormal number of signals pose problems in diagnostic terms. The inter-observer variability comes from the various criteria for defining a “spot” (i.e. of the fluorescent signal), for example its size, its intensity and its texture, but also due to the fact that the spots are often in various focal planes, according to the vertical axis Z in the nucleus.

The results can therefore differ significantly according to these criteria, which themselves depend on the preparation of the sample in each laboratory. A considerable bias is constituted by the establishing of the “cut-off”, i.e. of the “positivity threshold” value after which the sample is considered to be abnormal (Ruangvutilert et al, 2000). This value is difficult to establish since the percentage of nuclei having three signals in the control cases can range between 10 and 20% of the nuclei analyzed. This figure varies according to studies, since the number of cells analyzed can itself range from 50 to 200, which explains this statistical variability. In addition, it may be important for the medical interpretation of this test to reliably know the value of the proportion of nuclei carrying a chromosome number abnormality (mosaic).

The aim of the present invention is to provide an automated method of detection which, by its very design, thus eliminates the difficulties associated with reading by a human operator, and provides a solution to the problems concerning the variability in the preparation of the samples, which, until now, affected the analysis thereof.

This aim is achieved with a method for detecting, using a computerized system, interchromosomal imbalance by fluorescent in situ hybridization (FISH) on interphase nuclei, comprising the following phases:
in situ hybridizing of fluorescent probes on two separate chromosomes,
visualizing each probe with a different fluorochrome,
measuring intensity signals corresponding, respectively, to each probe thus visualized, on a set of nuclei, this measurement being carried out, firstly, in a control cell population and, secondly, in a cell population subjected to detection,
calculating the ratio between said signals corresponding, respectively, to said probes, this ratio calculation being carried out, firstly, on said control cell population so as to provide a reference ratio and, secondly, on said cell population subjected to detection,
comparing the ratio between signals corresponding to the cell population subjected to detection with the reference ratio, and
processing the result of this comparison in order to detect an interchromosomal imbalance.

For the purpose of the present invention, the term “in situ hybridization” is intended to denote a technique for locating a DNA (or RNA) sequence by means of a probe of a specific sequence homologous to that studied. It is based on nucleotide complementarity (A/T, A/U, G/C); it can be carried out under precise physicochemical conditions on chromosomal, cell or tissue preparations. The result of the in situ hybridization process is the formation of a hybrid between a probe and a target. In situ hybridization includes a denaturation step, the actual step of in situ hybridization of the probe on the target, and a step for detecting the hybrid or the probe. In the context of fluorescent in situ hybridization, the probes are labeled with a fluorophor and the hybridization is visualized by fluorescent labeling. The recent development of this technique enables the simultaneous visualization, on the same preparation, of several probes, each visualized with a different fluorophor.

The measurement of two or more signals corresponding respectively to each probe is advantageously carried out by image cytometry. Two probes can, for example, be visualized respectively with a green fluorochrome and with a red fluorochrome.

An approach by image cytometry has thus been developed which combines the advantages of analysis by FISH on interphase nuclei with those of signal reading which is rapid, precise, quantitative and objective, since it is independent of the operator.

This approach consists in using chromosome paints or paints for chromosomal regions, firstly, for the chromosome exhibiting an imbalance and, secondly, for another chromosome (autosome or sex chromosome), the latter being chosen such that the hybridization signal obtained with the chromosome paint is comparable to that obtained with the painting of the chromosome exhibiting an imbalance. In practice, the reference chromosome and the chromosome exhibiting an imbalance are, as far as possible, similar in size. Thus, for example, the present approach consists in using chromosome paints or paints for chromosomal regions, firstly, for chromosome 21 and, for example, secondly, for chromosome 20 or chromosome 22, chromosome 20 or 22 being taken as a reference since the hybridization signal obtained with painting of chromosome 21 is comparable to that obtained with painting of chromosome 20 or chromosome 22, which are similar in size. For chromosome 13 aneuploidy (for example: trisomy 13), the reference chromosome is preferably chosen from chromosomes 14 and 15. For a chromosome 18 aneuploidy (for example, trisomy 18), the reference chromosome is preferably chosen from chromosomes 17, 19 and 20. For X chromosome aneuploidy, the reference chromosome is, for example, chosen from chromosomes 6, 7, 8, 9, 10, 11 and 12. For Y chromosome aneuploidy, the reference chromosome is preferably chosen from chromosomes 19, 20, 21 and 22.

According to a preferred embodiment, the probes used in the in situ hybridization are chromosome painting probes. The term “chromosome painting probe” or “chromosome paint” is intended to denote a probe or a composition of probes which is suitable for hybridizing, under the hybridization conditions, with a target which comprises a predetermined chromosome of a multichromosomal genome. If only a fraction of such a chromosome is present in the sample subjected to such a hybridization with such a composition of probes, then this fraction hybridizes and is identified. In practice, the painted probe can be mixed with a second, a third, etc., so as to allow simultaneous labeling and detection of two, or three, etc., predetermined chromosomes. Various painting probes are currently commercially available (GIBCO-BRL; ONCOR; BOEHRINGER-MANHEIM; etc.). They are prepared by IRS-PCR amplification (see, for example, WO 00/22164), by DOT-PCR amplification using degenerate PCR primers for chromosomes, or from fragments of chromosomes isolated by chromosome sorting, by flow cytometry or by microdissection of chromosomes. Alternatively, the probes used in the present invention are probes covering the centromeric region (α-satellite DNA), or all or part of a chromosomal arm (telomeric sequences, for example TTAGGₙ), or specific for repeat DNA sequences which are specific for one or more chromosomes (conventional satellite DNA 1, 2 and 3).

According to a preferred embodiment, the chromosome painting probes are labeled with a nonisotopic entity such as luminescent agents, dyes, and the like. This labeling can be carried out indirectly by labeling the probes with enzymes, biotin, avidin, streptavidin, digoxigenin, haptens and the like, which will be visualized with luminescent agents or dyes. The luminescent agents, depending on the source of excitation energy, can be classified as radioluminescent, chemiluminescent, bioluminescent and photoluminescent (including fluorescent and phosphorescent). The term “fluorescent” generally refers to the property of a substance (such as a fluorophor) of producing light when it is excited by an energy source such as ultraviolet light, for example. More preferably, the probes are labeled with a fluorophor.

In the present invention, the inventors propose to detect an interchromosomal imbalance in any animal or human interphase cells, preferably in fetal cells, originating from amniotic fluid or from maternal blood. The detection of constitutional or acquired (cancers) chromosomal imbalances can also be carried out, for example, in the peripheral blood cells, in the cells of the skin, in the buccal cells and, more generally, in any cell type readily available for carrying out a diagnostic test.

In the present invention, the inventors propose to detect an interchromosomal imbalance, in place of detection of the number of copies of the chromosome analyzed:
after in situ hybridization of the probes for chromosomes 21 and 22 (for example), each probe is visualized with a different fluorochrome (in green or in red);
using a computerized system of image cytometry, the intensities of each probe are measured on a minimum of a few hundred (for example, 500) nuclei, after they have been recognized by the system, within a control cell population and a test population;
the calculation of the ratio between the two intensity signals from each of the probes makes it possible, by comparing it with the normal reference case, to determine the presence of at least one supernumerary chromosome (chromosome 21, for example). The interchromosomal imbalance is then greater than 1, and is 1.5 in the case of a trisomy. Calculation of the ratio between the two intensity signals from each of the probes also makes it possible, by comparing it with the normal reference case, to determine the presence of chromosomal monosomy. The interchromosomal imbalance would then be 0.5.

The advantages of the method of detection according to the invention, in comparison with the current methods of detection, are as follows:
due to the fact that the FISH technique is used, the method of detection according to the invention makes it possible to avoid all the disadvantages associated with cell culture, and makes it possible to obtain the result more rapidly (in a few days) and on a number of cells which is both statistically significant and not restrictive for samples with a low number of cells (between 500 and a few thousand cells).
All the difficulties associated with reading and interpretation by a human operator disappear with this method, since the analysis is carried out automatically by a computerized system; in particular, the recognition of the interphase nuclei, in which the measurement of the intensities of total fluorescence associated with the hybridization of each probe makes the analysis much simpler and much more robust than that using the recognition and counting of fluorescent signals currently proposed (“spot counting” in Tkachuk et al., 1991).

Moreover, even though the use of small probes gives signals with better resolution for counting, said signals are then more readily confused with the background noise, and the signal to noise ratio is therefore lower, all the more so since amniotic fluids contain clouds of protein, a considerable source of background noise. The inventors' approach, using chromosome paints, provides a higher signal to noise ratio than the methods using counting of small signals (counting of centromeric probes, in particular), by virtue of the size of the signal obtained.

The evaluation and the subtraction of the background noise make it possible to detect the trisomic cells under conditions which are difficult due to the characteristics of amniotic samples.

In addition, the problems concerning the variability of the preparation of the samples no longer affect the analysis. A possible change in the experimental conditions would not change the fluorescence ratio, since the two signals will be affected in the same way. It is possible to detect the presence of two populations within a cell population with better reliability by virtue of better statistical precision, due to the fact that it is possible to rapidly analyze a greater number of nuclei. This makes it possible to distinguish cases with maternal cell contamination or mosaic cases (cases where only a fraction of the fetal cells show the abnormality).

Finally, the particular cases comprising isochromosomes 21 or t translocations (21; 21) (Stoll et al, 1998) are detected with better reliability, whatever the nature of the rearranged chromosome. The use of chromosome paint shows a spot which should double in size and in intensity; measurement with an adapted camera remains more precise given the size of the signal. This approach is capable of being applied to the other pathologies concerning chromosomes 13 and 18 and the sex chromosomes, but also other pathologies where chromosome number imbalances are present (monosomy, trisomy, quadrisomy, etc., polysomy). It should be noted that the fluorescence in situ hybridization FISH technique has already been used in a different method, disclosed in the patent application published under the number FR2783253, for detecting an intrachromosomal imbalance in interphase nuclei, more precisely for detecting a loss or a gain of genetic material (deletion, insertion, amplification, duplication, etc.) of all or part of a chromosomal arm. In this method of detection, two probes which are labeled with two different fluorochromes and are respectively specific for the long arm and for the short arm of a chromosome are hybridized on this same chromosome.

In a particularly advantageous embodiment of the method according to the invention, the measuring phase comprises a first step consisting in acquiring, in a defined measuring plane, a given number of fields so as to obtain, for a given image magnification, at least a predetermined number (several hundreds or thousands) of analyzable nuclei.

The acquisition step also comprises the capturing, by means of successive narrow-bandpass optical filtering, of several images corresponding, for each field of observation, respectively to a plurality of planes corresponding to the wavelengths of a plurality of fluorochromes (for example: 4,6-diamidino-2-phenylindole (DAPI) with a blue fluorescence, fluorescein isothiocyanate (FITC) with a green fluorescence and Texas Red™ with a red fluorescence), storage of these acquired images and superimposition of said acquired and stored images.

According to another aspect of the invention, a system is proposed for detecting chromosomal region imbalances by fluorescent in situ hybridization of interphase nuclei, using the method according to the invention, comprising:
means for carrying out a fluorescent in situ hybridization on two separate chromosomes,
means for visualizing each probe with a different fluorochrome,
a device for measuring intensity signals corresponding respectively to each probe thus visualized, on a set of nuclei, firstly, within a control cell population and, secondly, within a cell population subjected to detection,
means for calculating the ratio between said signals corresponding respectively to said probes, firstly, on said control cell population so as to provide a reference ratio and, secondly, on said cell population subjected to detection,
means for comparing the ratio between signals corresponding to the cell population subjected to detection with the reference ratio, and
means for processing the result of this comparison for the purpose of detecting an interchromosomal imbalance.

The image cytometry device advantageously comprises:
multiple fluorescence microscopy means applied to cell populations subjected beforehand to a fluorescent in situ hybridization,
means for acquiring sets of images produced by the fluorescence microscopy means,
means for storing said acquired images, and
means for analyzing said acquired and stored images.

The fluorescence microscopy means and the image acquisition means cooperate so as to acquire, in a defined measuring plane, a given number of fields so as to obtain, for a given image magnification, at least a predetermined number of analyzable nuclei.

In a preferred configuration of a system of detection according to the invention, said system also comprises filtering means which cooperate with the fluorescence microscopy means and the acquisition means in order to acquire several images corresponding, for each field, respectively to a plurality of planes corresponding to the wavelengths of a plurality of fluorochromes (for example: DAPI, FITC (green), Texas Red (red)).

Moreover, the fluorescence microscopy means and the acquisition means can advantageously cooperate so as to acquire, within the same plane of analysis, images corresponding to a control cell population and images corresponding to a cell population subjected to detection.

Other advantages and characteristics of the invention will emerge on examination of the detailed description of an embodiment which is in no way limiting, and of the attached drawings in which:

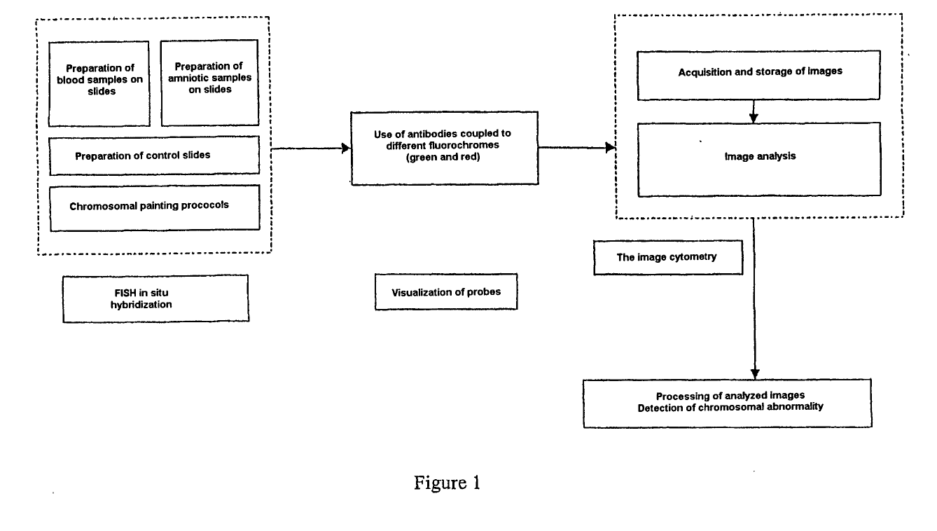

FIG. 1 illustrates the main phases of the method for detecting chromosomal imbalance according to the invention;

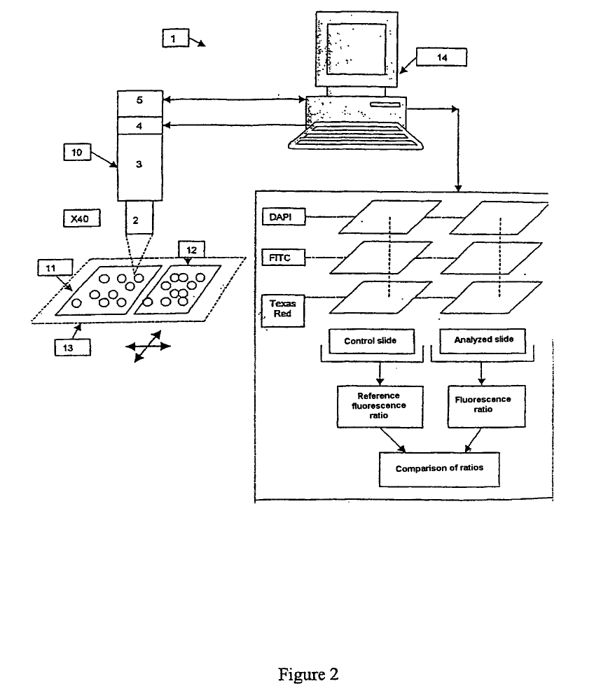

FIG. 2 represents diagrammatically the structure of a system of detection according to the invention; and

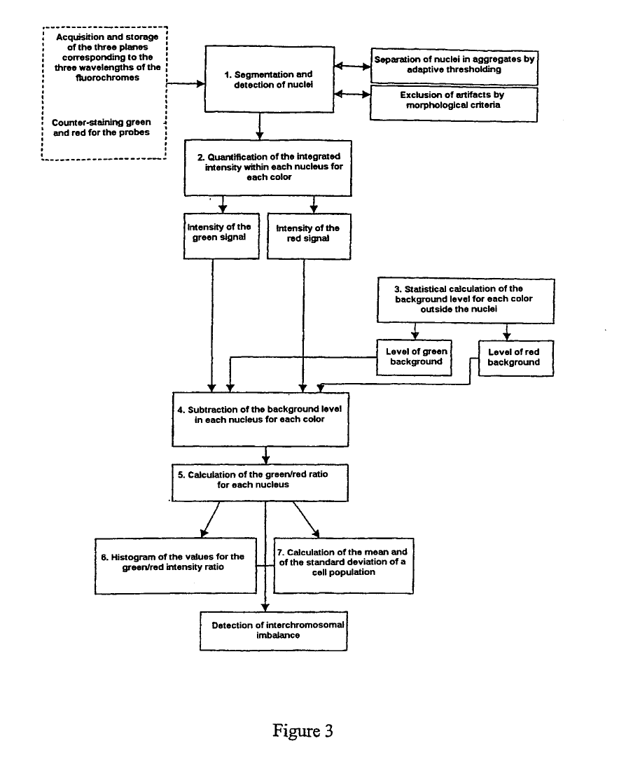

FIG. 3 is a flowchart representative of the detection and quantification operations carried out on the images acquired by the system of detection according to the invention.

The initial phases of preparation and of hybridization which precede the detection and quantification phase will firstly be described, in the context of an example of implementation of the method of detection according to the invention, in particular with reference to FIG. 1.

Sample Preparation

The test samples, currently derived from amniotic fluid, are prepared as for a conventional cytogenetic analysis on a slide, whether this involves samples placed in culture or analyzed directly. However, in order to improve the automatic analysis of the slides, the use of a centrifuge with wells for spot-plating on slides (for example a cytospin™) is recommended for flatplating of the cells. This makes it possible to reduce the number of cells “out of focus” during the automatic reading. The limited plating also decreases the image acquisition time.

The cultured amniotic samples are treated according to conventional protocols for prenatal diagnosis. For the direct fluid analysis, treatment with pepsin is followed by treatment with cell aggregate-dissociating medium.

The plating out is also carried out using a centrifuge with wells. The control slides can be prepared from normal amniotic fluids or from normal fibroblasts according to the same protocol. In addition, the analysis can also be performed on blood samples. In the case of a sample derived from maternal peripheral blood, the detection of fetal cells is carried out beforehand.

Probe Preparation

The in situ hybridization can be carried out according to the usual protocols for chromosome paints (Truong et al, 1998). The slides are first incubated for 15 minutes in a 2SSC saline solution, followed by rinsing with PBS buffer and treatment with pepsin in 0.01 N HCl (4 μg/ml, SIGMA) for 10 minutes. The digestion is stopped with a bath of PBS for 5 minutes, and the slides are fixed for 10 minutes in Carnoy's fixative (ethanol/acetic acid, 3:1 (V/V)) and air-dried.

The probe is then denatured for 10 minutes at 70° C. in 70% formamide (FLUKA)/2SSC at pH 7. The target DNA, i.e. the nuclei on the slides, is denatured under the same conditions for 3 minutes. The slides are then rinsed with 2SSC and dehydrated in a series of alcohol baths. The probe is hybridized overnight at 37° C. Post-hybridization washes are performed at 72° C. for 5 minutes in 2SSC buffer.

In the case of the use of digoxigenin-labeled or biotin-labeled probes, the detection of the probes is carried out using antibodies coupled to different fluorochromes (green and red).

In the case of the use of probes directly labeled with fluorochromes, the slides can be stained with DAPI (1 μg/ml, Molecular Probes) and mounted in p-phenyldiamine straightaway.

Method for Detecting and Quantifying the Fluorescent Signals from the Probes

The image cytometry device 1, used in the system for detecting interchromosomal imbalance according to the invention, comprises, with reference to FIG. 2, an acquisition component 10 including a fluorescent microscope 2, 3, a cooled, black and white CCD camera 5 allowing integration times ranging from 40 ms to 10 ms, a device (motorized filter wheel or motorized filter turret) for automatically changing the fluorescent probe excitation and emission light spectra 4 placed between the lamp and the microscope 3 and, optionally, between the microscope 3 and the CCD camera 5, and an X- and Y-motorized sample slide platform 13, automatically controlled by the device 1 for systematic sampling, and a control and processing component 14 consisting of a computer comprising, in particular, an image acquisition board.

The measurement of fluorescence is broken down into two steps:

1) Image acquisition and storage, after definition of a region of analysis on the slide studied.

2) Analysis of images and processing of the data which are derived therefrom.

The first step consists in acquiring, within the limits of a defined surface area, a given number of fields, with the objective allowing, for example, a 40× magnification, so as to obtain 500 to 1000 analyzable nuclei. The images are acquired by means of a CCD camera using selective interferential optical filters. For each field, three image planes corresponding to the wavelengths of the various fluorochromes (blue, green, red) are stored and superimposed (FIG. 2). Reference points are established in order to avoid the observed fields being out of focus. Thus, the slides subjected to detection 11 and the control slide 12 are analyzed under the same conditions (in particular, same integration time and same camera gain).

During the second step, which includes detection of the nuclei and quantification of the signals, the following is carried out, with reference to FIG. 3:

1) Segmentation of each image and detection of the nuclei to be analyzed, including separation of nuclei in aggregates, and also the exclusion of artifacts by criteria of size and morphology, making it possible to establish a mask of nuclei on the counter-staining plane, for example DAPI plane, in which the fluorescence intensities are measured.

2) Quantification of the integrated intensity within each nucleus for each fluorochrome (red and green), corresponding to the fluorescence emitted by each of the probes used, without any hypothesis regarding the form and the location of the signals:

3) Quantification of the background level for each color and each field outside the nuclei; determination of the most common noise level as reference.

4) Subtraction of the background level (reference value) in the nuclei for each of the probes.

5) Calculation of the green/red intensity ratio, corrected for background level, for each nucleus.

6) Representation of the data, for example in the form of a histogram or of a “scatterplot” (two-dimensional representation of the intensity values for the two colors).

7) Automatic determination of the mean and of the standard deviation of the fluorescence ratio values for a population of nuclei chosen by cluster analysis, i.e. by a method of analysis of clouds of points and determination of the centers of gravity of these clouds of points.

The fluorescence ratio is calculated for each sample in the same way and under the same conditions. Trisomy 21 is thus detected when the fluorescence ratio for the sample from the patient, normalized with respect to the control sample, is 1.5.

Of course, the invention is not limited to the examples which have just been described, and many arrangements can be introduced into these examples without departing from the context of the invention. The method of detection according to the invention can thus be extended to other chromosomes: X, Y, 13 and 18 or else other less recurrent abnormalities.

The system of detection is characterized in that:
the image cytometry device also comprises means for detecting nuclei and quantifying fluorescence signals of multiple wavelengths;
the detection and quantification means are organized so as to segment nuclei on the basis of an analysis based on multiple criteria of shape, size, fluorescence density, usually referred to as morphometric and densitometric criteria, giving rise to the creation of a mask for all the fields of a measuring plane.

### References

Eiben B, Trawicki W, Hammans W, Goebel R, Pruggmayer N, Epplen J T. Rapid prenatal diagnostics of aneuploidies in uncultured amniocytes by fluorescence in situ hybridization. Fetal Diagn Ther 1999; 14:193-197.
Pierluigi M, Perfumo C, Cavani S, Lehrach H, Nizetic D, Dagna Bricarelli F. An improved method for the detection of Down's syndrome aneuploidy in uncultured amniocytes. Clin Genet 1996; 49:32-36.
Ruangvutilert P, Delhanty J D, Rodeck C H, Harper J C. Relative efficiency of FISH on metaphase and interphase nuclei from non-mosaic trisomic or triploid fibroblast cultures. Prenat Diagn 2000; 20:159-162.
Soloviev I V, Yurov Y B, Vorsanova S G, Fayet F, Roizes G, Malet P. Prenatal diagnostics of trisomy 21 using interphase fluorescence in situ hybridization of post-replicated cells with site-specific cosmid and cosmid contig probes. Prenat. Diag 1995; 15: 237-248.
Steinborn A, Röddiger S, Born H J, Baier P, Halberstadt E. Fluorescent-in-situ-hybridization allows rapid prenatal detection of fetal aneuploidies within few hours. Z Geburtsh Neonatol 1996; 200:186-190.
Stoll C, Alembik Y, Dott B, Roth M P. Recent trends in the prevalence of down syndrome in north-eastern France. Ann Génét 1994; 37: 179-183.
Stoll C, Alernbik Y, Dott B, Roth M P. Study of Down Syndrome in 238942 consecutive births. Ann. Génét 1998; 41:44-51.
Thein A T A, AbdelFattah S A, Kyle P M, Soothill P W. An assessment of the use of interphase FISH with chromosome specific probes as an alternative to cytogeneticists in prenatal diagnostics. Prenat Diagn 2000; 20:275-280.
Tkachuk D C, Pinkel D, Kuo W. L, Weier A-U, Gray J. W, Clinical applications of fluorescence in situ hybridation. Genetic analysis techniques and applications 1991 April No. 2 New York, US.
Van Opstad D, van Hemel J O, Eussen B H, van der Heide A, van den Berg C, In ‘T Veld P A, Los F J. A chromosome 21-specific cosmid cocktail for the detection of chromosome 21 aberrations in interphase nuclei. Prenat Diagn 1995, 15:705-711.
Verma L, Macdonald F, Leedham P, MacConachi M, Dhanjai S, Hulten M. Rapid and simple prenatal DNA diagnostics of Down's syndrome. Lancet 1998; 352:9-12.
Wei H J, Su T H, Chien C L, Tzeng C R. Fluorescence in situ hybridization (FISH) as a method to detect aneuploid cells. Fetal Diagn Ther 1997; 12:309-312.

1. A method for detecting interchromosomal imbalance by fluorescent in situ hybridization on interphase nuclei, comprising the following phases:
in situ hybridizing of fluorescent probes on two separate chromosomes,
visualizing each probe with a different fluorochrome,
measuring intensity signals corresponding, respectively, to each probe thus visualized, on a set of nuclei, this measurement being carried out, firstly, in a control cell population and, secondly, in a cell population subjected to detection,
calculating the ratio between said signals corresponding, respectively, to said probes, this ratio calculation being carried out, firstly, on said control cell population so as to provide a reference ratio and, secondly, on said cell population subjected to detection,
comparing the ratio between signals corresponding to the cell population subjected to detection with the reference ratio, and
processing the result of this comparison in order to detect an interchromosomal imbalance.

2. The process as claimed in claim 1, characterized in that the measurement of the two signals corresponding respectively to each probe is carried out by automated image cytometry.

3. The process as claimed in either of claims 1 and 2, characterized in that the two probes are visualized, respectively, with two different fluorochromes (for example, a green fluorochrome and a red fluorochrome).

4. The method as claimed in any one of the preceding claims, characterized in that the intensity signals from each probe are measured on a number of nuclei which can be defined by the operator, for example a few hundred.

5. The method as claimed in any one of the preceding claims, characterized in that the measuring phase comprises a first step consisting in acquiring, in a defined measuring plane, a given number of fields so as to obtain, for a given image magnification, at least a predetermined number of analyzable nuclei.

6. The method as claimed in claim 5, characterized in that the acquisition step also comprises acquisition, by means of successive optical filtering, of several images corresponding, for each field, respectively to a plurality of planes corresponding to the wavelengths of a plurality of fluorochromes (for example, blue for the counter-staining, green and red for the probe labeling), storage of said acquired images and superimposition of said acquired and stored images.

7. The method as claimed in either of claims 5 and 6, characterized in that the acquisition step is carried out under acquisition conditions which are substantially identical for a control cell population and a cell population subjected to detection.

8. The method as claimed in claim 7, characterized in that the acquisitions corresponding respectively to the control cell population and cell population subjected to detection are carried out on two fields included in the same measuring plane.

9. The method as claimed in one of claims 5 to 8, characterized in that the measuring phase also comprises a second step for detecting nuclei and quantifying fluorescence intensity signals.

10. The method as claimed in claim 9, characterized in that the detection and quantification step comprises segmentation of the nuclei in each field included in a measuring plane.

11. The method as claimed in claim 10, characterized in that the segmentation of the nuclei includes separation of nuclei in aggregates.

12. The method as claimed in either of claims 10 and 11, characterized in that the segmentation of the nuclei includes elimination of the artifacts by criteria of morphology and size.

13. The method as claimed in any one of claims 9 to 12, characterized in that the detection and quantification step comprises quantification of the integrated fluorescence signal intensity within each nucleus for each color corresponding to each probe.

14. The method as claimed in claim 13, characterized in that the detection and quantification step also comprises calculation of the background level for each color outside the nuclei in each of the fields.

15. The method as claimed in claim 14, characterized in that the detection and quantification step also comprises determination of the most common background level as noise reference level and correction with said reference level of the fluorescent signal intensity quantified within each nucleus.

16. A system for detecting chromosomal imbalances by fluorescent in situ hybridization on interphase nuclei, using the method as claimed in any one of the preceding claims, comprising:
means for carrying out a fluorescent in situ hybridization on two separate chromosomes,
means for visualizing each probe with a different fluorochrome,
a device for measuring intensity signals corresponding respectively to each probe thus visualized, on a set of nuclei, firstly, within a control cell population and, secondly, within a cell population subjected to detection,
means for calculating the ratio between said signals corresponding respectively to said probes, firstly, on said control cell population so as to provide a reference ratio and, secondly, on said cell population subjected to detection,
means for comparing the ratio between signals corresponding to the cell population subjected to detection with the reference ratio, and
means for processing the result of this comparison for the purpose of detecting an interchromosomal imbalance, even in the case of a mosaic.

17. The system of detection as claimed in claim 16, characterized in that it includes, as measuring means, an image cytometry device.

18. The system of detection as claimed in claim 17, characterized in that the image cytometry device comprises:
multiple fluorescence microscopy means applied to cell populations subjected beforehand to a fluorescent in situ hybridization,
means for acquiring images produced by the fluorescence microscopy means,
means for storing said acquired images, and
means for analyzing said acquired and stored images.

19. The system of detection as claimed in claim 18, characterized in that the fluorescence microscopy means and the image acquisition means cooperate so as to acquire, in a defined measuring plane, a given number of fields so as to obtain, for a given image magnification, at least a predetermined number of analyzable nuclei.

20. The system of detection as claimed in claim 19, characterized in that it also comprises filtering means which cooperate with the fluorescence microscopy means and the acquisition means in order to acquire several images corresponding, for each field, respectively to a plurality of planes corresponding to the wavelengths of a plurality of fluorochromes (for example: DAPI (blue) for the counter-staining, FITC (green) and Texas Red™ (red) for the probes).

21. The system of detection as claimed in one of claims 18 to 20, characterized in that the fluorescence microscopy means and the acquisition means cooperate so as to acquire, within the same plane of analysis, images corresponding to a control cell population and images corresponding to a cell population subjected to detection.

22. The system of detection as claimed in one of claims 17 to 21, characterized in that the image cytometry device also comprises means for detecting nuclei and quantifying fluorescence signals of multiple wavelengths.

23. The system of detection as claimed in claim 22, characterized in that the detection and quantification means are organized so as to segment nuclei on the basis of a morphometric and densitometric analysis, giving rise to the creation of a mask for all the fields of a measuring plane.

24. The system of detection as claimed in claim 22 or 23, characterized in that the detection and quantification means are organized so as to separate the nuclei in aggregates.

25. The system of detection as claimed in one of claims 22 to 24, characterized in that the detection and quantification means are organized so as to exclude artifacts by criteria of size and morphology.

26. The system of detection as claimed in one of claims 22 to 25, characterized in that the detection and quantification means are organized so as to quantify the integrated fluorescent signal intensity within each nucleus for each color.

27. The system of detection as claimed in one of claims 22 to 26, characterized in that the detection and quantification means are organized so as to calculate the background level for each color outside the nuclei.

28. The system of detection as claimed in one of claims 22 to 27, characterized in that the detection and quantification means are organized so as to determine the most common background level as background reference value and to subtract said reference value from the intensity quantified within each nucleus.

29. The application of the method for interchromosomal detection as claimed in any one of claims 1 to 15, to nuclei of fetal cells circulating in maternal blood.
